(19) United States
(12) Patent Application Publication
(10) Pub. No.: US 2014/0000616 A1
(43) Pub. Date: Jan. 2, 2014

(54) PATIENT INTERFACE DEVICE HAVING A FABRIC POUCH FOR A CUSHION

(71) Applicant: Richard Thomas Haibach, Verona, PA (US)

(72) Inventor: Richard Thomas Haibach, Verona, PA (US)

(73) Assignee: KONINKLIJKE PHILIPS N.V., EINDHOVEN (NL)

(21) Appl. No.: 14/004,912

(22) PCT Filed: Mar. 13, 2012
(86) PCT No.: PCT/IB2012/051171

§ 371 Date: Sep. 13, 2013

Related U.S. Application Data
(60) Provisional application No. 61/452,218, filed on Mar. 14, 2011.

(51) Int. Cl. A61M 16/06; A61M 16/08; A61M 16/00

(52) U.S. Cl. CPC A61M 16/0683; A61M 16/0057; A61M 16/0875; A61M 16/0666
USPC 128/205.25; 128/204.18

(57) ABSTRACT

A system is structured to provide a regimen of respiratory therapy to a patient having an airway. The system includes a gas flow generator structured to generate a flow of breathing gas, a patient interface device structured to communicate the flow of breathing gas to the airway of the patient, and a patient circuit structured to couple the gas flow generator to the patient interface device. The patient interface device includes headgear, a cushion structured to communicate the flow of breathing gas to the airway of the patient, and a fabric pouch holding the cushion. The fabric pouch is supported by or coupled to the headgear.

### CROSS-REFERENCE TO RELATED APPLICATIONS

This patent application claims the priority benefit under 35 U.S.C. §119(e) of U.S. Provisional Application No. 61/452,218 filed on Mar. 14, 2011, the contents of which are herein incorporated by reference.

### BACKGROUND OF THE INVENTION

1. Field of the Invention

The present invention relates to respiratory patient interface devices, and, in particular, to patient interface devices having a cushion, such as for example and without limitation, a pillows cushion. The invention also relates to systems employing a patient interface device to provide a regimen of respiratory therapy.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. Such patient interface devices may also employ other patient contacting components, such as forehead supports, cheek pads and chin pads. The patient interface device is connected to a gas delivery tube or conduit and interfaces a ventilator or pressure support device with the airway of the patient, in order that a flow of breathing gas can be delivered from a pressure/flow generating device to the airway of the patient. It is known to maintain such devices on the face of a patient by headgear having one or more straps adapted to fit over/around the patient's head.

Adherence and compliance to therapy, such as CPAP or other pressure support therapies, is growing to be an industry-wide issue. Factors such as comfort and ease of use of a patient interface device can greatly affect a patient's adherence and compliance to therapy. Thus, easier to use, simplified designs for patient interface devices are becoming expectations for any product that seeks to compete.

### SUMMARY OF THE INVENTION

In accordance with one aspect of the invention, a patient interface device is structured to communicate a flow of breathing gas to an airway of a patient. The patient interface device comprises: headgear, a cushion structured to communicate the flow of breathing gas to the airway of the patient, and a textile or fabric pouch holding the cushion, wherein the fabric pouch is supported by or coupled to the headgear.

As another aspect of the invention, a system is structured to provide a regimen of respiratory therapy to a patient having an airway. The system comprises: a gas flow generator structured to generate a flow of breathing gas; a patient interface device structured to communicate the flow of breathing gas to the airway of the patient, the patient interface device comprising: headgear, a cushion structured to communicate the flow of breathing gas to the airway of the patient, and a fabric pouch holding the cushion, wherein the fabric pouch is supported by or coupled to the headgear; and a patient circuit structured to couple the gas flow generator to the patient interface device.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is an isometric view of a system structured to provide a regimen of respiratory therapy to a patient according to embodiments of the present invention, with some components shown schematically for ease of illustration;

FIG. 2 is an isometric view of a low profile fabric mask in accordance with an embodiment of the invention;

FIG. 3A is an exploded isometric view of the low profile fabric mask of FIG. 2;

FIG. 3B is an exploded top plan view of portions of the fabric of the low profile fabric mask of FIG. 2 prior to assembly;

FIG. 3C is an isometric view of the assembled portions of the fabric of FIG. 3B;

FIG. 4 is an isometric view of portions of a low profile fabric mask in accordance with another embodiment of the invention;

FIG. 5 is an isometric view of a low profile fabric mask in accordance with another embodiment of the invention;

FIG. 6 is an exploded isometric view of portions of the low profile fabric mask of FIG. 5;

FIG. 7 is an isometric view of a portion of the fabric of the low profile fabric mask of FIG. 5;

FIGS. 8-11 are isometric views of a low profile fabric mask in accordance with another embodiment of the invention;

FIG. 12 is an isometric view of a pillows cushion that provides a non-adjustable angle with respect to the patient's nose in accordance with another embodiment of the invention; and

FIGS. 13-15 are isometric views of a pillows cushion that provides an adjustable angle with respect to the patient's nose in accordance with another embodiment of the invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

As used herein, the word “fabric” means a material consisting of a network of natural and/or artificial fibers made by, for example and without limitation, interlacing, weaving, knitting, spreading, crocheting, or bonding the fibers to form the network. Non-limiting examples of a flexible fabric material include fabric materials such as Lycra® (or another type of spandex material), silk, or polyester, or a laminate of different materials (e.g., without limitation, a fabric material such as Lycra®, a foam intermediate layer, and a unbroken loop such as Velcro®). Other non-limiting examples of fabric material include a portion of a fabric material has a laminated plastic sheet to provide structure, or fabric headgear includes a relatively more rigid section at or near an area corresponding to a patient's cheek.

As used herein, the word “headgear” means any suitable device structured to mount a patient interface device to the head of a patient. Non-limiting examples of headgear include a number of fabric members, a number of silicone members, or a number of strap members structured to mount a patient interface device to the head of a patient. Other non-limiting examples of such members can include a laminated plastic sheet to provide structure, or a relatively more rigid section at or near an area corresponding to a patient's cheek.

As used herein, the word “cushion” means a nasal mask or nasal cushion that covers the nose of a patient; a nasal cushion or pillows cushion having nasal prongs that are received within the nares of a patient (e.g., without limitation, a nasal cannula); or a nasal saddle type cushion structured to be placed against an underside portion of the nose of a patient and to engage and cover the nares of the patient.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

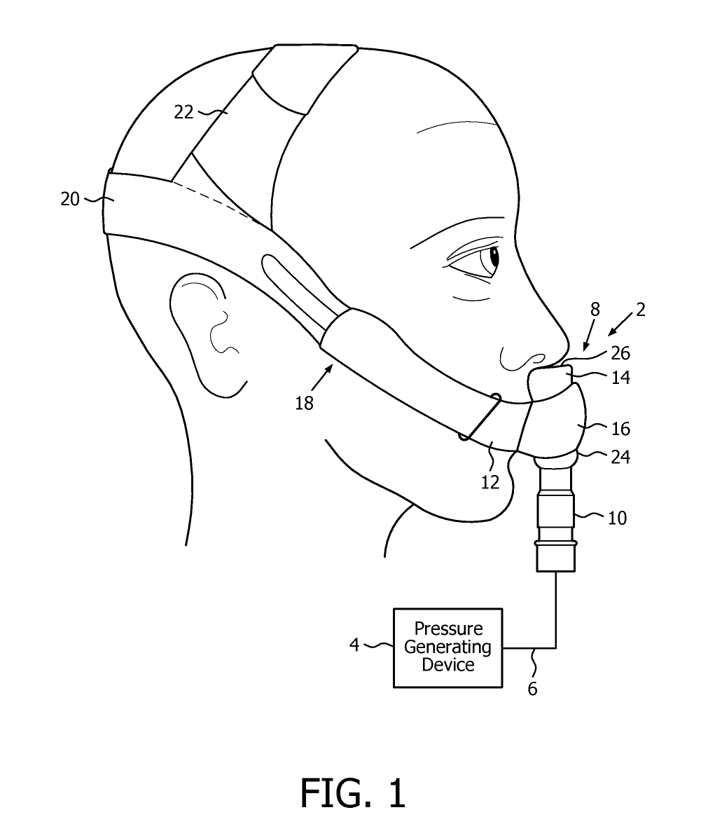

FIG. 1 shows an example system 2 structured to provide a regimen of respiratory therapy to a patient having according to one example embodiment. For example, system 2 may be configured to provide a CPAP therapy or other sleep apnea therapy (e.g., OSA therapy) to a patient. System 2 includes a pressure generating device 4, a patient circuit 6, and a patient interface device 8 having a fluid coupling conduit 10 (e.g., without limitation, an elbow conduit). A gas flow generator, such as pressure generating device 4, is structured to generate a flow of breathing gas and may include, without limitation, ventilators, constant pressure support devices (such as a continuous positive airway pressure device, or CPAP device), variable pressure devices (e.g., without limitation, BiPAP®, Bi-Flex®, or C-Flex™ devices manufactured and distributed by Philips Respironics of Murrysville, Pa.), and auto-titration pressure support devices. Patient circuit 6 is structured to couple pressure generating device 4 to patient interface device 8 and communicate the flow of breathing gas from pressure generating device 4 to patient interface device 8. Patient circuit 6 typically includes a gas delivery conduit or tube coupled to fluid coupling conduit 10.

Patient interface device 8 is structured to communicate the flow of breathing gas to the patient's airway (e.g., the patient's nares in this example). Patient interface device 8 includes a fabric member 12 (e.g., without limitation, any suitable fabric, cloth or textile coupled to or part of headgear), a cushion 14 structured to communicate the flow of breathing gas to the airway of the patient, and a fabric pouch 16 holding cushion 14. Fabric pouch 16 is supported by fabric member 12.

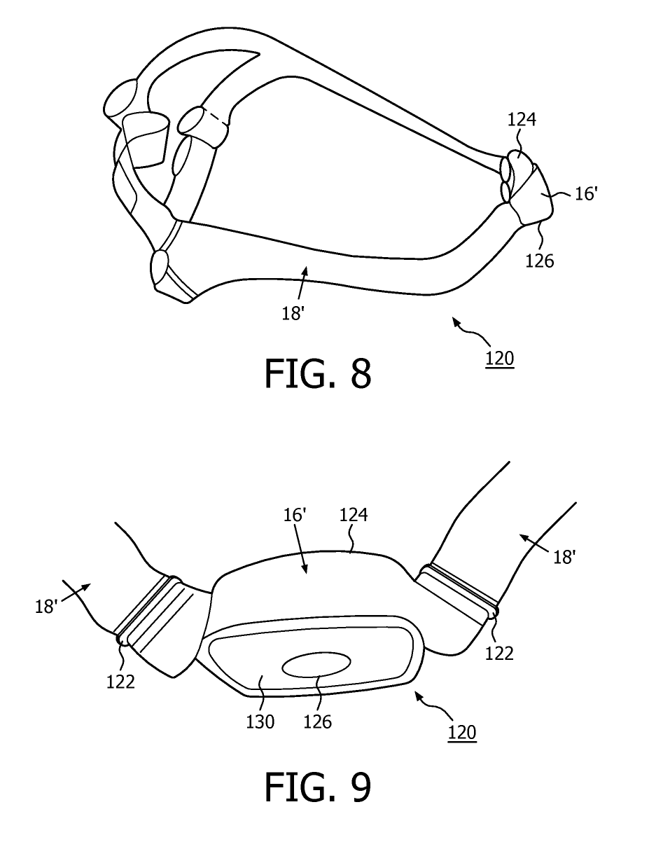

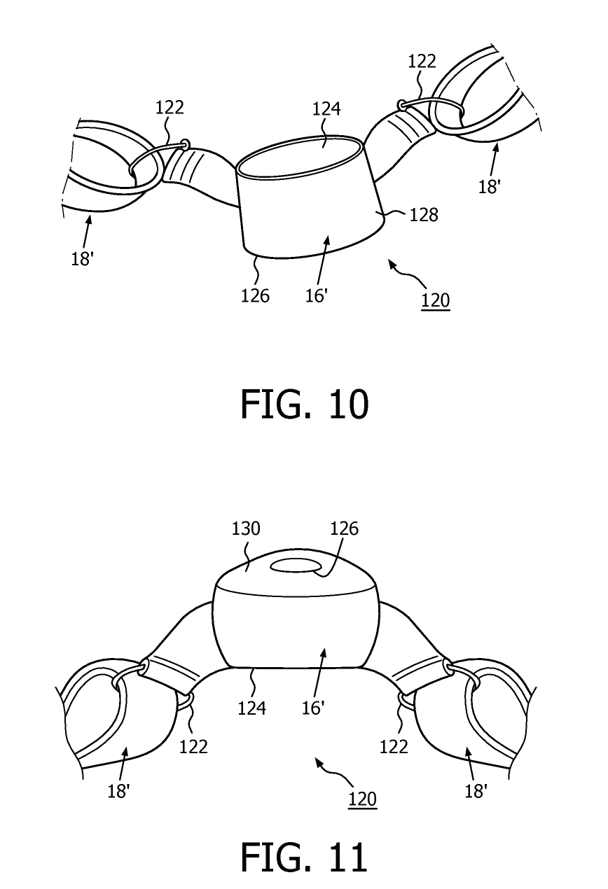

In the example of FIG. 1, fabric member 12 couples to or forms headgear 18 and is structured to mount patient interface device 8 to the head of the patient. As will be described below in connection with FIGS. 3B and 7, fabric member 12 can include fabric that forms fabric pouch 16. In this example, fabric pouch 16 is part of headgear 18. Alternatively, as will be described below in connection with FIGS. 8-11, fabric pouch 16′ can be coupled to headgear 18′. As is conventional, portions, such as a number of straps 20,22, of headgear 18 are structured to secure patient interface device 8 to the head of the patient.

In the illustrated embodiment, patient interface device 8 is a nasal cushion having nasal prongs that are received within the patient's nares. Any type of patient interface device, however, such as a nasal saddle type cushion structured to be placed against the lower, underside portion of the nose of a patient (wherein the patient's nares are engaged and covered), or a nasal mask that covers the nose, which facilitates the delivery of the flow of breathing gas to, and the removal of a flow of exhalation gas from, the airway of such a patient may be used while remaining within the scope of the present invention.

In the embodiment shown in FIG. 1, patient interface device 8 includes cushion 14, which is held by fabric pouch 16. An opening 24 of fabric pouch 16 to which fluid coupling conduit 10 is coupled allows the flow of breathing gas from pressure generating device 4 to be communicated to an interior space defined by cushion 14, and then, to the airway of the patient. Opening 24 of fabric pouch 16 also allows the flow of exhalation gas (from the airway of the patient) to be communicated to an exhaust port (not shown in FIG. 1, but see FIGS. 3A, 4, 6 and 12) that may be provided, for example and without limitation, in fluid coupling conduit 10, in cushion 14, in fabric pouch 16, or elsewhere on patient interface device 8. Example patient interface device 8 further includes headgear 18 configured to secure patient interface device 8 to the patient's head. As will be described in greater detail below, in the example embodiment, headgear 18 comprises a single piece wrap-around fabric member structured to be wrapped around the head of the patient such that cushion 14 is held in place within fabric pouch 16 in a manner that permits gases to be communicated to and from the airway of the patient through cushion 14.

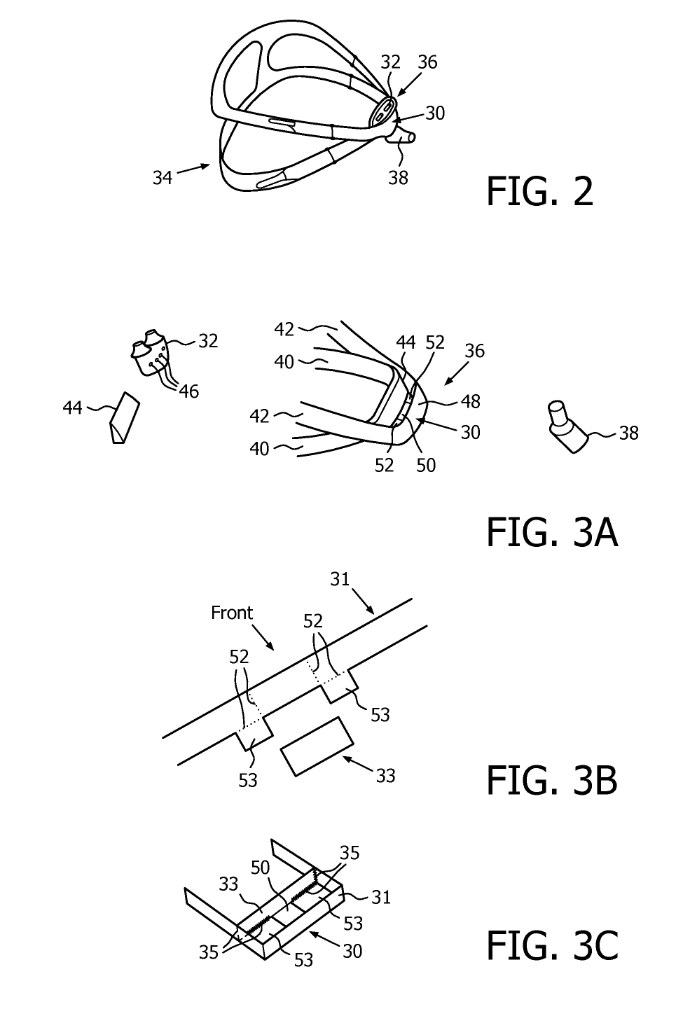

Referring to FIGS. 2 and 3A, a low profile fabric pouch 30 houses a pillows cushion 32, and provides, for example and without limitation, CPAP therapy to a patient (not shown, but see FIG. 1). Fabric pouch 30 mounts to headgear 34 or, alternatively, could be part of headgear 34. Fabric pouch 30 and cushion 32 form a low profile patient interface device 36 that helps to reduce torque thereon as the patient moves around. A ball and socket attachment 38 further reduces torque. The reduced size and footprint of patient interface device 36 is aesthetically pleasing. Patient interface device 36 is composed almost entirely of soft materials, for improved comfort, simplicity, and appeal.

FIGS. 3A-3C shows various structures employed to form and cooperate with fabric pouch 30 (best shown in FIG. 3C). These structures include fabric straps 40, 42 of headgear 34, a number of optional removable wedges 44 (e.g., without limitation, two are shown, which can be made from lightweight foam or other material or combination of materials, with enough rigidity to hold a shape, yet be comfortable) to adjust the angle with respect to the nose of the patient, pillows cushion 32, and ball and socket attachment 38. Exhalation from the patient to pillows cushion 32 and to various opening 46 thereof is diffused through a portion 48 of fabric pouch 30, in order to reduce noise and jetting.

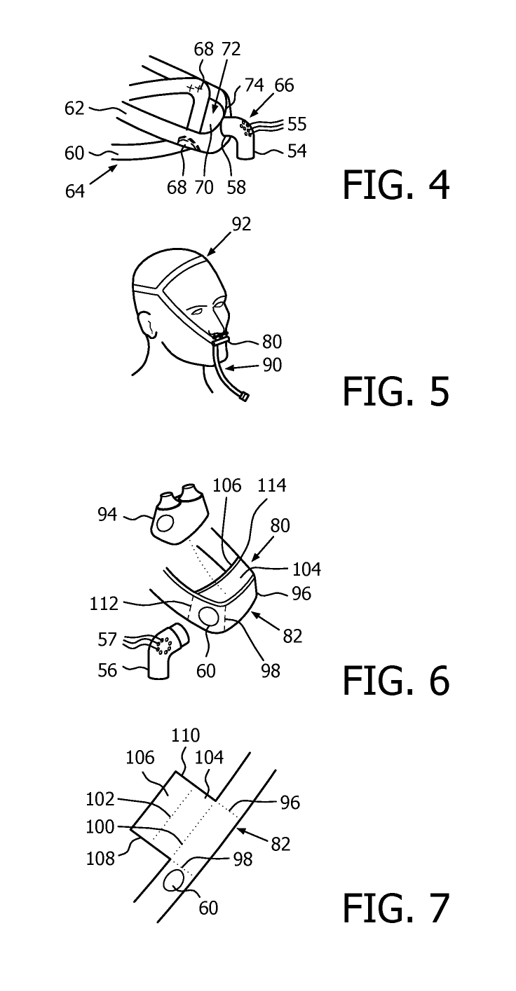

Ball and socket attachment 38 is coupled to pillows cushion 32 through a lower (with respect to FIG. 3A) opening 50 (best shown in FIG. 3C) in fabric pouch 30 in order to reduce torque on pillows cushion 32 applied by a patient circuit (not shown, but see patient circuit 6 of FIG. 1) (e.g., without limitation, a hose). Fabric pouch 30 comprises a number of pieces of fabric 31,33, a plurality of folds 52, a plurality of tabs 53 and a plurality of stitches 35. Ball and socket attachment 38 (or a suitable elbow connector 54 of FIG. 4 or 56 of FIG. 6) couples pillows cushion 32 to a patient circuit and passes through opening 50 of fabric pouch 30 (e.g., after pillows cushion 32 is assembled in fabric pouch 30 with optional number of removable wedges 44). Although a lower or bottom opening 50 is shown in FIG. 3A, a front opening 58 (FIG. 4) or a side opening 60 (FIG. 6) can be employed. Ball and socket attachment 38 (FIG. 3A) (or elbow connectors 54 (FIGS. 4) and 56 (FIG. 6)) is disposed proximate pillows cushion 32 in order to reduce torque on pillows cushion 32 applied by a patient circuit.

Patient interface device 36 eliminates the need for rigid frame components, which could come in contact with the face of the patient and reduce comfort. Patient interface device 36 also stays tight to the face and has a minimal footprint. Patient interface device 36 has fabric pouch 30 to hold, for example and without limitation, somewhat standard pillows cushion 32. Fabric pouch 30 is entirely supported by fabrics, such as 31,40,42, to mount it to the head of the patient.

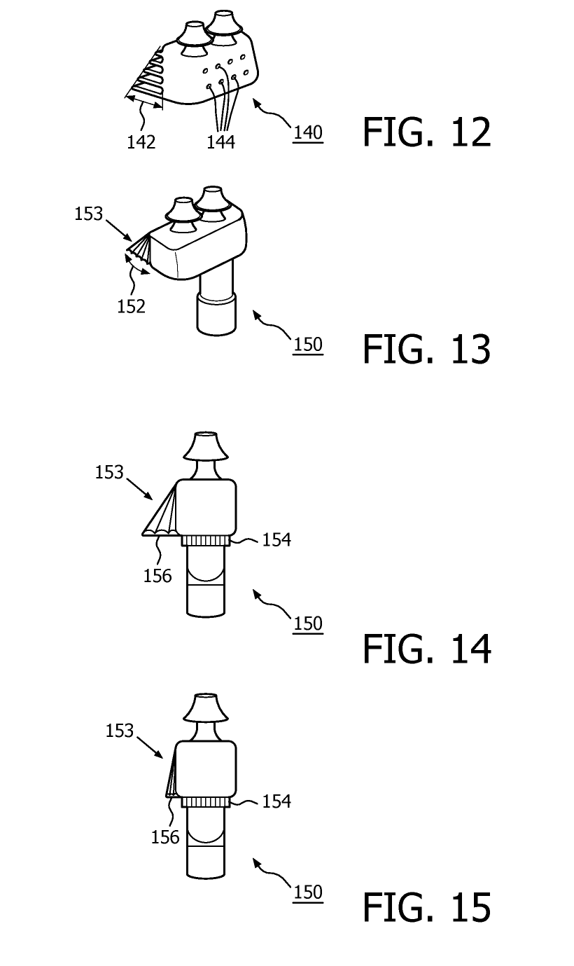

If optionally employed, number of removable wedges 44 can adjust the angle with respect to the nose of the patient, although this angle could be built into pillows cushion 32 (as will be discussed, below, in connection with FIG. 12), or included in a self-contained rotating mechanism (as will be discussed, below, in connection with FIGS. 13-15).

Pillows cushion 32 can be molded from silicone. Fabric pouch 30 for pillows cushion 32 can be built with a series of folds and sewn, as shown in FIGS. 3A-3C.

FIG. 4 shows that portions 60, 62 (e.g., without limitation, a number of straps) of nasal mask headgear 64 are configured to secure patient interface device 66 to the head of a patient (see the patient of FIG. 1). A number of portions 68 can be stitched (e.g., without limitation, two side stitched areas) or could pass through a slot (not shown) in back (with respect to FIG. 4) strap 60. This allows other strap 62 to slide through strap 60 as it is tightened. Elbow connector 54 passes through a through hole 58 in the fabric of strap 62, after pillows cushion (not shown, but see pillows cushion 32 of FIG. 3A) is assembled. This helps ensure that the assembly of patient interface device 66 will stay together. In an exemplary embodiment, elbow connector 54 remains suitably close to pillows cushion 32, in order to reduce the torque applied by a hose (not shown, but see patient circuit 6 of FIG. 1). In a further exemplary embodiment, any fabric of straps 60, 62 touching below the nose of the patient remains soft, and comfortable to the touch. Straps 60, 62 and a bottom (with respect to FIG. 4) piece of fabric 70 are stitched together to define a fabric pouch 72 having a first opening 74 structured to receive pillows cushion 32 (FIG. 3A) and second opening 58 structured to receive elbow connector 54 from a patient circuit after pillows cushion 32 is assembled.

The portions of fabric pouch 72 that contact the face of the patient can be built with a relatively softer fabric, if desired, and outer fabric can be relatively less elastic than inner softer fabric, which hugs and cushions the face of the patent. Opening 58 for elbow connector 54 and the corresponding air delivery can be located anywhere, but are likely located on the front, side or bottom of fabric pouch 72. Conventional elbow connector 54 can include conventional exhalation vents 55.

Fabric pouch 72 can be built with a plurality of pieces of fabric 60, 62, 70 as shown in FIG. 4. Alternatively, as shown in FIGS. 5-7, fabric pouch 80 can be built with a single piece of fabric 82.

FIGS. 5-7 show another patient interface device 90 including fabric pouch 80 having side opening 60 for conventional elbow connector 56 with exhalation vents 57. Fabric pouch 80 mounts, for example and without limitation, to headgear 92. Headgear 92 can be separate from or part of fabric pouch 80 that holds pillows cushion 94. FIG. 7 shows single piece of fabric 82 including four examples folds 96, 98, 100, 102. Front folds 96,98 (with respect to FIGS. 5 and 6) are shown in FIG. 6. An example generally rectangular panel 104 forms a bottom (with respect to FIG. 6) of fabric pouch 80, and another generally rectangular panel 106 forms a back (with respect to FIG. 6) of fabric pouch 80). Sides 108, 110 of panel 106 are stitched to corresponding respective portions 112, 114 of single piece of fabric 82.

FIGS. 8-11 show another low profile fabric mask 120 including fabric pouch 16′ coupled to headgear 18′ by couplers 122 (FIGS. 9-11). Somewhat similar to fabric pouch 16 of FIG. 1, fabric pouch 16′ includes a first opening 124 to receive a pillows cushion (not shown, but see pillows cushion 32 of FIG. 3A) and a second opening 126 (best shown in FIGS. 9 and 11) to receive a conventional elbow connector (not shown) or a ball and socket attachment (not shown). Fabric pouch 16′ can be formed from an outer ring of fabric 128 defining opening 124 and a bottom (with respect to FIG. 9) piece of fabric 130 having opening 126 and stitched to ring of fabric 128.

FIG. 12 shows a pillows cushion 140 providing a non-adjustable angle 142 with respect to the nose of the patient. A plurality of exhalation vents 144 function like vents 46 of FIG. 3A. Here, however, pillows cushion 140 can be disposed in fabric pouch 30 without any of optional wedges 44.

FIGS. 13-15 show a pillows cushion 150 providing an adjustable angle 152 with respect to the nose of the patient. Here, pillows cushion 150 includes a foldable structure 153 controlled by a twist knob 154 that adjusts a cable 156 to retract (fold) (FIG. 15) and deploy (unfold) (FIG. 14) foldable structure 153.

This invention could be applied primarily to sleep apnea masks, but could also be used in the critical care market. It can thus be appreciated that the present invention provides a patient interface device that can accommodate for issues relating to one or more of comfort, size/weight, sizing, and ease of use.

In the claims, any reference signs placed between parentheses shall not be construed as limiting the claim. The word “comprising” or “including” does not exclude the presence of elements or steps other than those listed in a claim. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device structured to communicate a flow of breathing gas to an airway of a patient, the patient interface device comprising:
headgear;
a cushion structured to communicate the flow of breathing gas to the airway of the patient; and
a fabric pouch holding the cushion, wherein the fabric pouch is supported by or coupled to the headgear,
wherein the fabric pouch is structured to hold a number of removable wedges to adjust an angle with respect to a nose of the patient.

2. The patient interface device of claim 1, wherein the patient includes the nose having nares, and wherein the cushion is selected from the group consisting of a pillows cushion, a nasal mask that covers the nose of the patient, a nasal cushion having nasal prongs that are received within the nares of the patient, and a nasal saddle type cushion structured to be placed against an underside portion of the nose of the patient and to engage and cover the nares of the patient.

3. The patient interface device of claim 1, wherein the headgear is structured to mount the patient interface device to a head of such a patient; and wherein the headgear includes fabric that forms the fabric pouch.

4. (canceled)

5. The patient interface device of claim 1, wherein the fabric pouch includes a first opening receiving the cushion and a second opening structured to receive an elbow connector from a patient circuit.

6. (canceled)

7. The patient interface device of claim 1, wherein the cushion as held by the fabric pouch defines one of a non-adjustable angle with respect to the nose of the patient, and an adjustable angle with respect to the nose of the patient.

8. The patient interface device of claim 1, wherein the fabric pouch comprises a plurality of pieces of fabric, a plurality of folds and a plurality of stitches.

9. A system structured to provide a regimen of respiratory therapy to a patient having an airway, the system comprising:
(a) a gas flow generator structured to generate a flow of breathing gas;
(b) a patient interface device structured to communicate the flow of breathing gas to the airway of the patient, the patient interface device comprising:
(1) headgear,
(2) a cushion structured to communicate the flow of breathing gas to the airway of the patient, and
(3) a fabric pouch holding the cushion, wherein the fabric pouch is supported by or coupled to the headgear; and
(c) a patient circuit structured to couple the gas flow generator to the patient interface device,
wherein the fabric pouch is structured to hold a number of removable wedges to adjust au angle with respect to a nose of the patient.

10. The system of claim 9, wherein the system is structured to provide CPAP therapy, a sleep apnea therapy, an OSA therapy.

11. The system of claim 9, wherein the headgear is fabric headgear, and wherein the fabric pouch is coupled to the fabric headgear or is part of the fabric headgear.

12. The system of claim 9, wherein portions of the headgear are adapted to secure the patient interface device to a head of such a patient.

13. (canceled)

14. The system of claim 9, wherein the patient circuit includes an elbow connector disposed proximate the cushion in order to reduce torque on the cushion applied by the patient circuit.

15. The system of claim 14, wherein the fabric pouch includes an opening, and wherein the elbow connector couples the cushion to the patient circuit and passes through the opening of the fabric pouch.

16. (canceled)

17. The system of claim 9, wherein the headgear is structured to mount the patient interface device to a head of such a patient, and wherein the headgear includes fabric that forms the fabric pouch.

18. (canceled)
